(19) United States
(12) Patent Application Publication
(10) Pub. No.: US 2013/0267683 A1
(43) Pub. Date: Oct. 10, 2013

(54) PLANT PROTEIN HYDROLYSATES

(71) Applicants: Pieter Berends, Zoznegg-Muhlingen (DE); Swen Rabe, Muehlingen (DE); Lutz Fischer, Stuttgart (DE); Ralf Berger, Hanover (DE); Diana Linke, Bad Rehburg (DE)

(72) Inventors: Pieter Berends, Zoznegg-Muhlingen (DE); Swen Rabe, Muehlingen (DE); Lutz Fischer, Stuttgart (DE); Ralf Berger, Hanover (DE); Diana Linke, Bad Rehburg (DE)

(73) Assignee: NESTEC S.A., Vevey (CH)

(21) Appl. No.: 13/995,429

(22) PCT Filed: Dec. 21, 2011
(86) PCT No.: PCT/EP2011/073635

§ 371 Date: Jun. 18, 2013

(30) Foreign Application Priority Data
Dec. 21, 2010 (EP) 10196255.3

(51) Int. Cl. C12P 21/06

(52) U.S. Cl. CPC C12P 21/06
USPC 530/370; 435/297.1; 435/68.1

(57) ABSTRACT

A membrane reactor for the manufacture of plant protein hydrolysates, the membrane reactor comprising a substrate vessel adapted to provide a plant protein substrate to an enzyme source, a continuously stirred reactor comprising the enzyme source, and an ultrafiltration module comprising a membrane with a molecular cut-off wherein the membrane is adapted to allow passage of the plant protein hydrolysate while retaining the enzyme.

### TECHNICAL FIELD

The invention relates to a hydrolysis of plant proteins to form plant protein hydrolysates. In particular, the invention relates to an apparatus and a use of the apparatus for the manufacture of the plant protein hydrolysates. The invention also relates to a method for the manufacture of the plant protein hydrolysates.

### BACKGROUND

Protein hydrolysates such as amino acids and peptides have applications in food technology. The protein hydrolysates are used for providing taste active ingredients to food products.

Protein hydrolysates are manufactured by hydrolysis of a protein. Protein hydrolysates can therefore include amino acids and peptides which are obtained by the hydrolysis of the protein.

The use of enzymes for the hydrolysis of the protein is a known procedure. The enzymes are usually mixed with the protein to form the protein hydrolysates in a batch procedure. However, the use of enzymes in the batch procedure can be prohibitive as the enzymes cannot be collected from the mixture, isolated and reused. Furthermore, the cost of the enzymes can be up to 50% of the cost of total raw materials. Therefore, the batch procedure for the hydrolysis of proteins has its drawbacks.

Ultra filtration (UF) is a process of separating small molecules such as amino acids and peptides from protein hydrolysate mixtures using membranes. The basis for the separation is size exclusion of molecules such that particles such as amino acids and peptides are retained on the membrane, while other constituents of the mixture such as salt and water pass through the membrane. Therefore, U facilitates amino acid and peptide protein concentration. UF nevertheless has drawbacks and the effectiveness of UF is strongly dependent on operating parameters and hydrolysate characteristics. The operating parameters can be, for example, trans-membrane pressure, membrane cut-off, tangential fluid velocity and system hydrodynamics. The hydrolysate characteristics can be, for example, pH, viscosity, particle size, and salt concentration. That is to say that current UF technology requires the manipulation of a number of factors which is complicated and cumbersome to maintain in order to achieve efficient separation and isolation of the protein hydrolysates.

Plant proteins are partly water-insoluble. The structure of plant proteins is relatively large. The diffusion of plant proteins into an immobilization matrix such as a bed of immobilized enzymes has not been contemplated or considered. Consequently, the effectiveness of immobilized enzymes for plant protein hydrolysis is poor.

Nevertheless, the use of immobilized proteases for the manufacture of protein hydrolysates from peptides is known. However, due to a lack of effectiveness the feasibility of such systems is still hindered (see for example Walsh, M., K., Immobilized enzyme for food applications, in Novel enzyme technology for food applications, R. Rastall, Editor, 2007, CRC Press LLC: Boca Raton, p. 60-84).

An object of the present invention is therefore to provide an apparatus and method for the manufacture of plant protein hydrolysates that goes at least part way to overcoming one or more of the above disadvantages, or at least provides a useful alternative.

### SUMMARY OF THE INVENTION

In a first aspect the invention relates to a membrane reactor for the manufacture of plant protein hydrolysates, the membrane reactor comprising:

a) a substrate vessel adapted to provide a plant protein substrate to an enzyme source,

b) a continuously stirred reactor comprising the enzyme source; and

c) an ultrafiltration module comprising a membrane with a molecular cut-off wherein the membrane is adapted to allow passage of the plant protein hydrolysate while retaining the enzyme.

In preferred embodiments of the invention, the membrane reactor further comprises:

d) a first circulation loop enabling a mixture of the plant protein substrate and enzyme source to be transferred from the continuously stirred reactor to the ultrafiltration module and at least some of the mixture to be returned to the continuously stirred reactor; and

e) a second circulation loop enabling the mixture received from the first circulation loop to be circulated through or over the membrane and at least some of the mixture to be returned to the first circulation loop.

In a second aspect the invention relates to a use of the membrane reactor in the manufacture of plant protein hydrolysates for food stuffs.

In another aspect the invention provides a method for the manufacture of plant protein hydrolysates for use in food, the method comprising:

a) providing a suspension of plant protein,

b) adding to the suspension of plant protein an enzyme to form a mixture such that plant protein hydrolysis occurs,

c) filtering the resulting mixture through an ultrafiltration module comprising a membrane with a molecular cut-off; and

d) collecting the filtrate comprising the plant protein hydrolysate for use as a food.

Preferably, step c) of the method comprises circulating the mixture between a continuously stirred reactor and an ultrafiltration module such that some of the mixture is returned from the ultrafiltration module to the continuously stirred reactor and some of the mixture is circulated through or over the membrane.

In a further aspect the invention provides a plant protein hydrolysate obtainable by the method of the invention, wherein the plant protein hydrolysate is a taste enhancing compound for use in foodstuffs.

### BRIEF DESCRIPTION OF THE DRAWINGS

FIG. 1 shows a schematic representation of an operating window for development of membrane reactor technology according to an aspect of the invention.

FIG. 2 shows a diagram of a set-up for enzymatic hydrolysis of plant proteins with a membrane reactor according to an aspect of the invention.

FIG. 3 shows relative enzyme activity [%] in fractions collected during testing of a ceramic membrane in a cross-flow filtration module membrane with a 5 nanometre cut-off with plant protein.

FIG. 4 shows temperature stability of glutaminase activity determined with 1-γ-Glutamyl-p-Nitroanilid hydrolysis assay (T 57±1° C. and pH 5.0±0.2).

FIG. 5 shows the temperature stability of protease activity during hydrolysis of wheat gluten over time determined with l-leucine-para-Nitroanalid assay in the presence of substrate (T 57±1° C. and pH 5.0±0.2).

FIG. 6 shows plant protein hydrolysate yield over time [g/L*h] in lab-scale enzyme membrane reactor experiments using 10 kDa, 5 kDa and 1 kDa molecular cut-off membranes according to aspects of the invention.

FIG. 7 shows increased release of amino acids from an enzyme membrane reactor compared to a batch reactor applying the same enzyme concentration and the same size of membrane reactor (50 L) (pH 5.0, T 50° C.).

FIG. 8 shows an amino acid profile of plant protein hydrolysate. The plant protein hydrolysates of the invention are a naturally balanced mixture of peptides and amino acids.

FIG. 9 shows a HPLC analysis that the plant protein hydrolysate of the membrane reactor according to the invention does not differ significantly from plant protein hydrolysate of the batch process.

FIGS. 10 to 14 show yield and enzyme stability results of processes using a double loop enzyme membrane system.

### DETAILED DESCRIPTION

In a first aspect the present invention relates to a membrane reactor for the hydrolysis of a plant protein to form plant protein hydrolysates. The membrane reactor combines advantages of enzyme immobilization (e.g. lower enzyme substrate ratio) and the enzyme batch system (e.g. good enzyme/substrate contact). The membrane reactor enables large scale hydrolysis of plant proteins to form plant protein hydrolysates.

The membrane reactor preferably comprises a double loop system. This system has two circulation loops. One loop operates at around atmospheric pressure and transfers a mixture of plant protein material and enzyme from a holding tank (or substrate vessel) to a second circulation loop. Most of the mixture passes to the second circulation loop, but some is circulated back to the holding tank in a continuous process. The mixture that passes to the second circulation loop is subjected to ultrafiltration. The second circulation loop operates under a pressure of 1 to 8 bar, preferably 6 bar, to force the mixture at high velocity (2 to 10 m/s) through or over the filtration membrane. The reason is to avoid the formation of a fouling layer of substrate on the membrane. The membrane has pores of suitable cut-off size (1-20 nm, preferably 5 nm) to enable the plant protein hydrolysate material of the invention to pass through the membrane (filtrate). Material that does not pass through the membrane (retentate) is recirculated in the second circulation loop.

The membrane reactor increases efficiency of plant protein hydrolysis. The efficiency is increased by re-usage of the enzyme's catalytic activity resulting in a better enzyme/plant protein ratio. Additionally, the removal of plant protein hydrolysate shifts the equilibrium of enzymatic action or microbial fermentation towards plant protein hydrolysate. Efficiency of plant protein hydrolysis is thus defined by the following three factors:
Space-time-yield [g/L/h]
Enzyme/plant protein hydrolysate ratio [nkat/g]
Plant protein/Plant protein hydrolysate ratio [%-w/w]

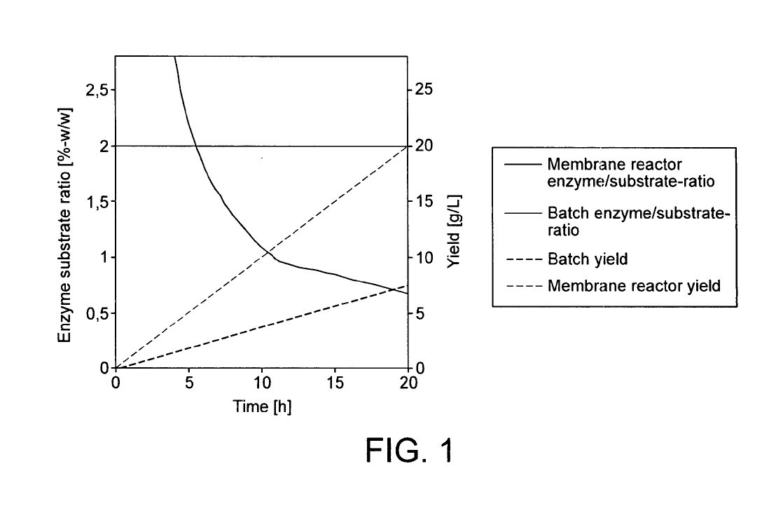

Therefore, process efficiency values of the batch process mean that an operating window for a semi-continuous membrane bioreactor system can be defined, from which the technological targets for the membrane reactor can be deduced. FIG. 1 represents the operating window for the development of a semi-continuous membrane bioreactor system, which is limited to 20 hours due to microbial stability of the enzymes. Furthermore, FIG. 1 shows a schematic representation of the operating window for the development of the membrane reactor technology to enzymatically hydrolyze the plant protein wheat gluten. Thus, from FIG. 1 it is determined that the enzyme:plant protein ratio (left y-axis, continuous line) must be below 2% w/w, as for example represented by the curved continuous line for a typical membrane bioreactor curve, which in this case goes through the break-even point at 6 hours. The space yield over time (right y-axis) must be above the lower dotted line, as for example shown by the upper dotted line.

The operating window as determined from FIG. 1 was a starting point for setting the experimental parameters in order to test feasibility of a method for manufacturing plant protein hydrolysates using the membrane reactor.

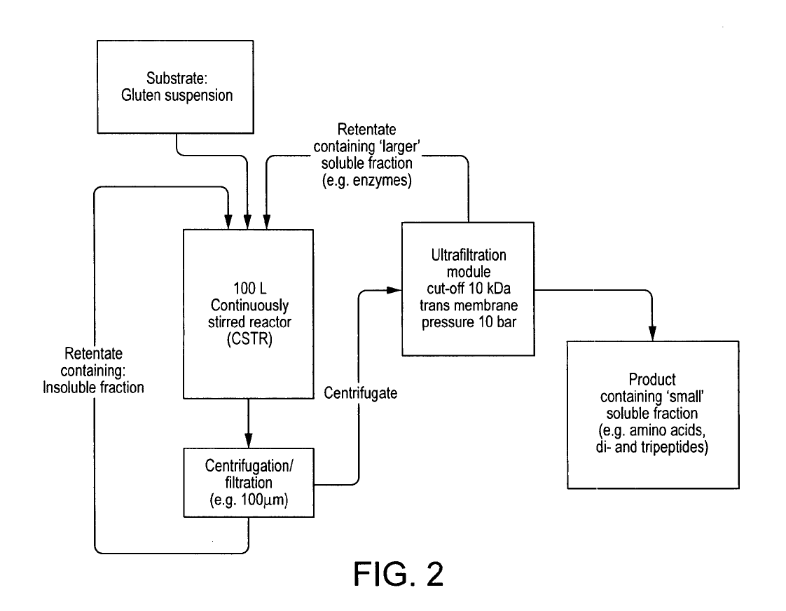

A schematic of an exemplary embodiment of the membrane reactor is shown in FIG. 2. A vessel for holding the substrate is also shown in FIG. 2. The substrate is a suspension of a plant protein, for example wheat gluten. The plant protein substrate is then fed to a continuously stirred reactor (CSTR) in which is also present the enzymes to form a mixture. The CSTR ensures a homogenous mixture of suspension of the plant protein and enzyme and therefore provides optimal conditions for hydrolysis of the plant protein to form the plant protein hydrolysates. Following hydrolysis, a separation of solid matter and liquid matter may be carried out. Any solid matter following the separation of solid matter and liquid matter is then returned to the CSTR. The resulting mixture containing the plant protein hydrolysate is then sent to an UF module. The UF module has a membrane with a molecular cut off (MCO). The membrane with the MCO determines which plant protein hydrolysates pass through the membrane. Different membranes can therefore be used. The UF module has a trans-membrane pressure (TMP) of 10 bar. The plant protein hydrolysates such as amino acids and peptides pass through the UF module and can be collected. A retentate that does not pass through the UF module is returned to the CSTR and the process repeats. It is to be understood that the membrane reactor is not a closed system and can be continuously replenished with more materials to form the plant protein hydrolysates.

An advantage of having separation of solid matter and liquid matter from the mixture from the CSTR prior to filtration is to avoid insoluble matter to foul and enter the membrane of the UF module. The separation of solid matter and liquid matter decreases the risk of fouling of the membrane and increases the output of plant protein hydrolysate. The separation of solid matter and liquid matter can be achieved by, for example, but not limited to, separation techniques such as centrifugation and metal edge filters as known in the art.

The membrane reactor can also include an electro dialysis system (not shown). The electro dialysis system operates by applying electrical potential difference through the membrane such that an electrical charge is passed over the membrane to cause diffusion of polar molecules such as amino acids through the membrane. The electro dialysis system enables a separation of the amino acids and the peptides from the plant protein hydrolysates.

According to an aspect of the invention the plant protein wheat gluten was mixed with water to obtain a suspension of plant protein of between 0.5 to 50% (w/w), preferably between 0.5% (w/w) to 22%, more preferably between 5 to 10% (w/w). It is observed that when the suspension of plant protein is between 0.5% (w/w) to 22% there is an improvement of pumping properties and a reduction of membrane fouling. In order to maintain stability of enzyme action the pH of the plant protein in water suspension is adjusted to pH 5 by the addition of acetic acid. Alternatively, to maintain stability of enzyme action the plant protein in water suspension is heated. Heating the plant protein in water suspension is preferred since the heating provides improved accessibility of the plant protein with the enzyme and enables a higher enzyme activity and microbial stability of the enzyme. The wheat gluten suspension is transferred to the continuously stirred reactor with a rate equal to a rate of formation of plant protein hydrolysate to ensure the continuous manufacture of plant protein hydrolysate. In the CSTR, the enzyme (or mixture of enzymes) 20-5000 nkat/L is present for hydrolysis of the plant protein to peptides and amino acids. The mixture entering the UF module is in cross-flow mode, circulated over a membrane (e.g. ceramic membrane) with a channel size that is large enough to avoid channel blockage by particles that are present in the mixture. A pore-size of the membrane of the UF module must be small enough to retain enzyme and plant proteins, but large enough to allow protein hydrolysates to pass through the membrane.

It is to be appreciated that following the manufacture of the plant protein hydrolysate, the plant protein hydrolysate can be dried.

The plant protein hydrolysates are useful for providing taste active ingredients to food products.

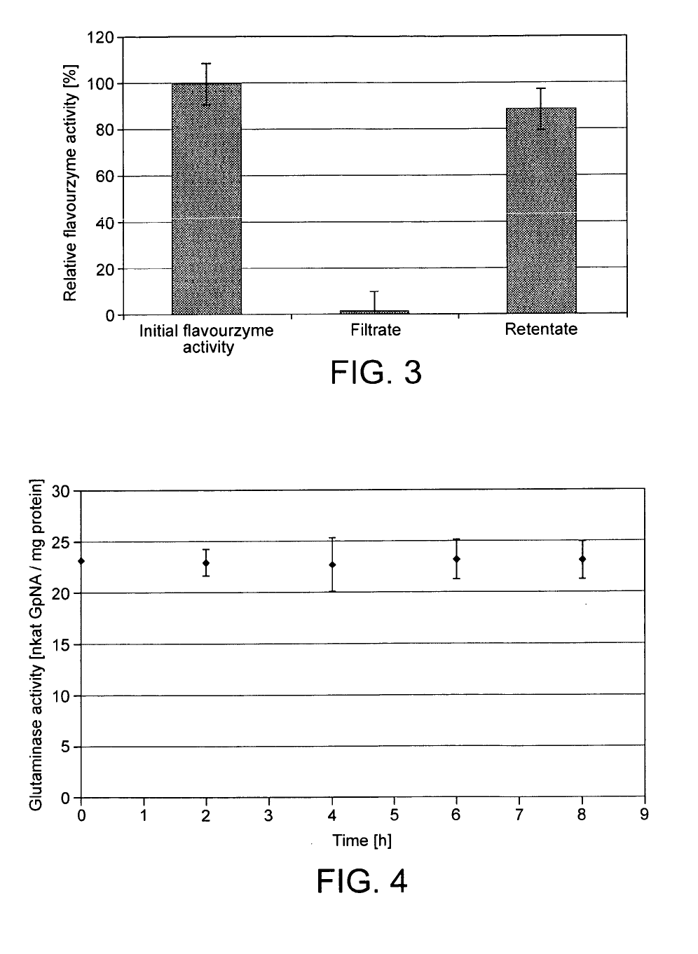

A ceramic membrane of the UF device with a 5 nanometre molecular cut-off pore size was tested for enzyme retention with plant protein. In various aspects of the invention the membrane of the UF device can have a molecular cut-off pore size of between 1 to 20 nanometres. The aim of the test was to assess a technical protease enzyme cocktail (Flavorzyme, [E] 264 nkat/L Leu-p-Na) passed though the membrane in the presence of plant protein (10% w/w wheat gluten). Enzyme retention by the membrane is important for the technical feasibility of the membrane bioreactor for plant protein hydrolysis. The results are shown in FIG. 3 in which it seen that no significant enzyme activity was lost over a time period of 3 hours.

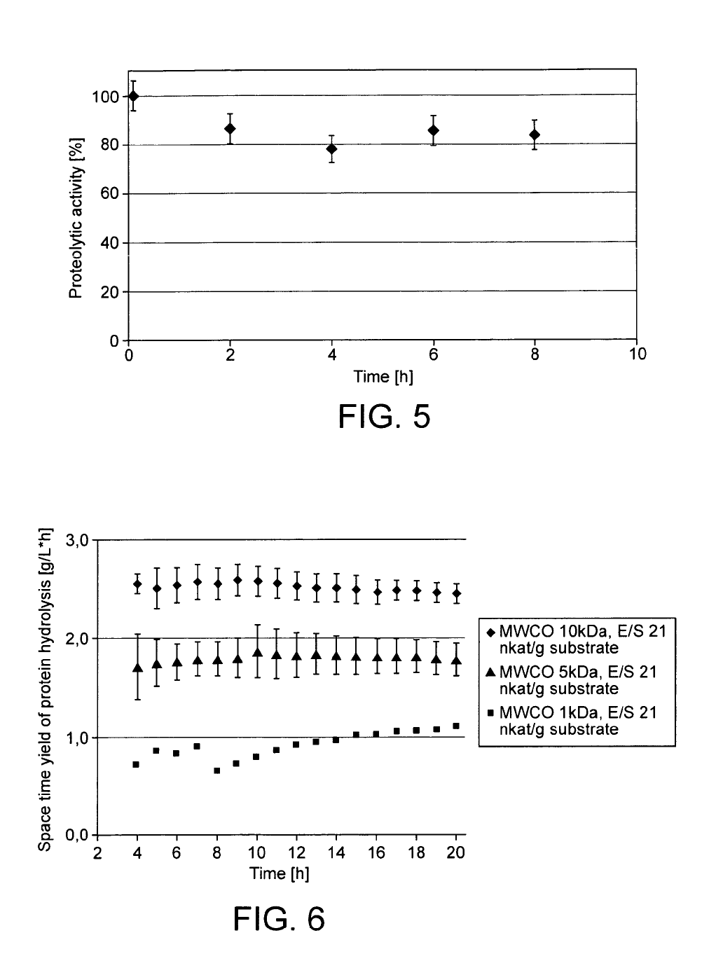

Furthermore, the stability of glutaminase activity was followed under process conditions (57±1° C. and pH 5.0±0.2 in the presence of substrate) by hydrolysis of the chromogenic substrate L-γ-Glutamyl-p-Nitroanilide (GpNA). The results of this stability investigation are shown in FIG. 4. Accordingly, glutaminase activity was stable over 8 hours processing time which is in accordance with earlier findings (see Mohamed i. Mahmoud, C.T.C., Protein Hydrolysates as Special Nutritional Ingredients. Novel Macromolecules in Food Systems, 2000: p. 181-215).

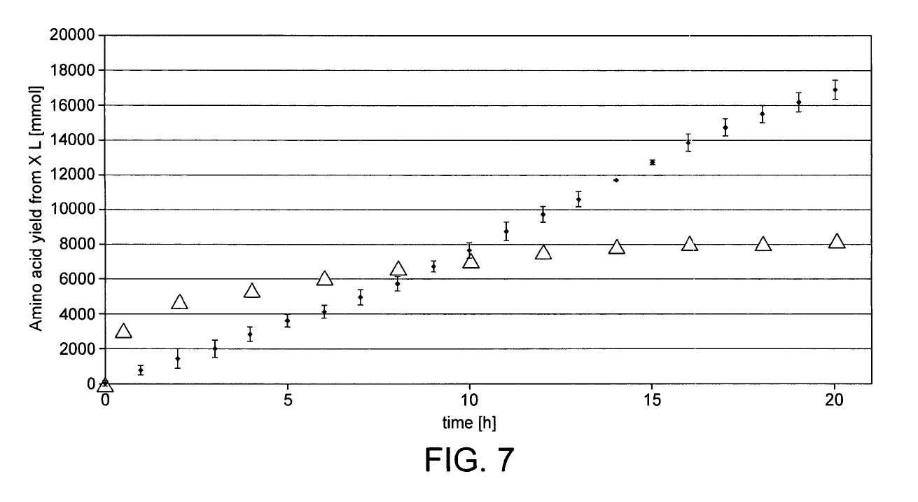

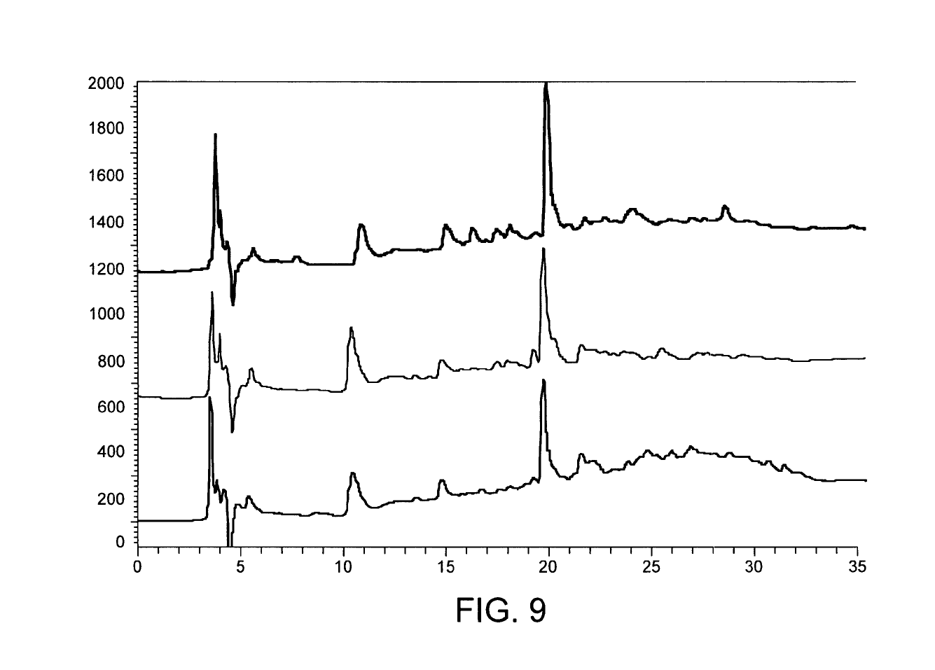

The enzyme activity of the protease enzyme Flavorzyme (available from Novozymes NS) was determined using the leucine para-nitroanilide method and wheat gluten substrate (see Deeslie, M.C.a.W.D., Soy Protein Hydrolysis in Membrane Reactors. JAOCS, 1983. 60(6): pp. 1112-1115). The initial activity was 264 nkat/L, FIG. 5 shows the relative activity of Flavorzyme over 8 hours at 57±1° C. and pH 5.0±0.2, measured using the leucine para nitroanilide method. After 24 hours at 57±1° C., the relative Flavorzyme activity was still 71±6%, which indicates a loss of enzyme activity per hour of slightly more than 1%. FIG. 5 demonstrates that the enzyme activity is stable under process conditions. The cause for a declined reaction rate (triangular line of FIG. 7) is product inhibition. By removing product via the membrane, product inhibition is evaded and the efficiency of the reactor and enzyme increases.

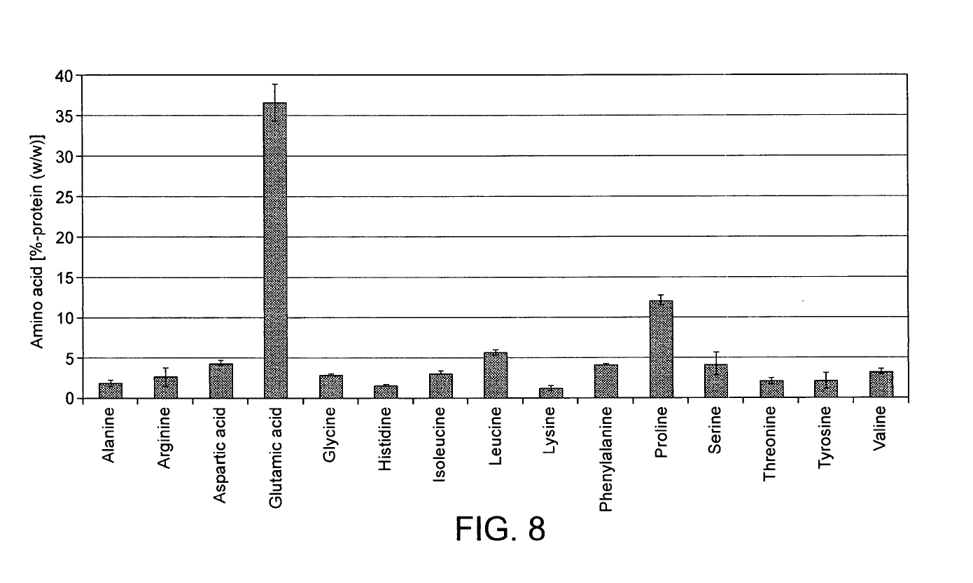

Laboratory tests with a membrane reactor using a 10 kDa filter and a wheat gluten substrate concentration of 0.5% (w/w) showed proof of principle of the membrane reactor for enzymatic wheat gluten hydrolysis. The product/substrate ratio of the membrane reactor was 51% since the amount of substrate used was 1 gram over 20 hours and the total product yield was 0.51 gram. The batch hydrolysis under the same conditions with the same absolute amount of enzyme (0.89 nkat/g), but a total reaction volume of 50 mL which correlates with a substrate amount of 0.25 gram resulted in a product/substrate ratio of 70% using a molecular weight cut-off (MWCO) of 10 kDa since the product yield was 0.18 gram. Therefore, according to the invention, the enzyme usage efficiency increased almost 3 times. The results are shown in FIG. 6. FIG. 6 shows in the top curve results using a MWCO of 10 KDa and an amount of enzyme/plant protein hydrolysate ratio (21 nkat/g). FIG. 6 shows in the middle curve results using a MWCO of 5 KDa and an amount of enzyme/plant protein hydrolysate ratio (21 nkat/g). FIG. 6 shows in the lower curve results using a MWCO of 1 KDa and an amount of enzyme/plant protein hydrolysate ratio (21 nkat/g).

The plant protein of the invention can of course be derived from other sources of plant protein aside from whey. The sources of plant protein can include, but are not limited to, plant protein derived from soy, corn, potato, pea or cassava.

The enzymes of the invention can be a single enzyme or a mixture of enzymes. The enzyme can be enzyme is at least one of an endopeptidase, an exopeptidase a glutaminsae and an enzyme derived from Basidiomycetes.

Also at pilot plant scale technical feasibility was evaluated. A significant improvement of amino acid release over time was shown when applying the same size of membrane reactor and the same enzyme concentration as shown in FIG. 7. Further optimization of operational conditions will result in even better amino acids yields.

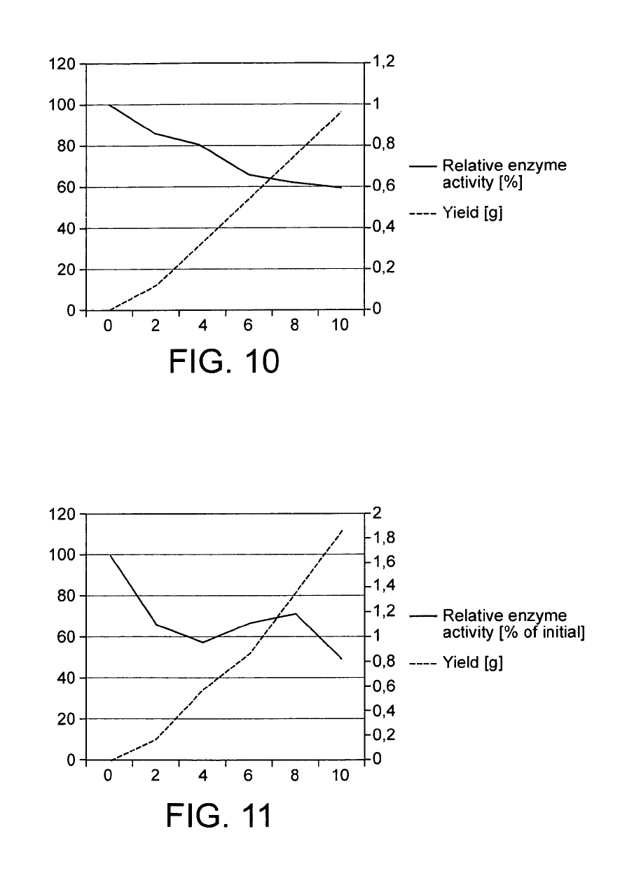

The amino acid profile of the plant protein hydrolysate is shown in FIG. 8. The amino acid profile of the plant protein hydrolysate was determined to comprise no residual plant protein, at least 10 percent of the peptides of the plant protein hydrolysate containing 2 to 5 amino acids, the amount of free amino acids being higher than 30%.

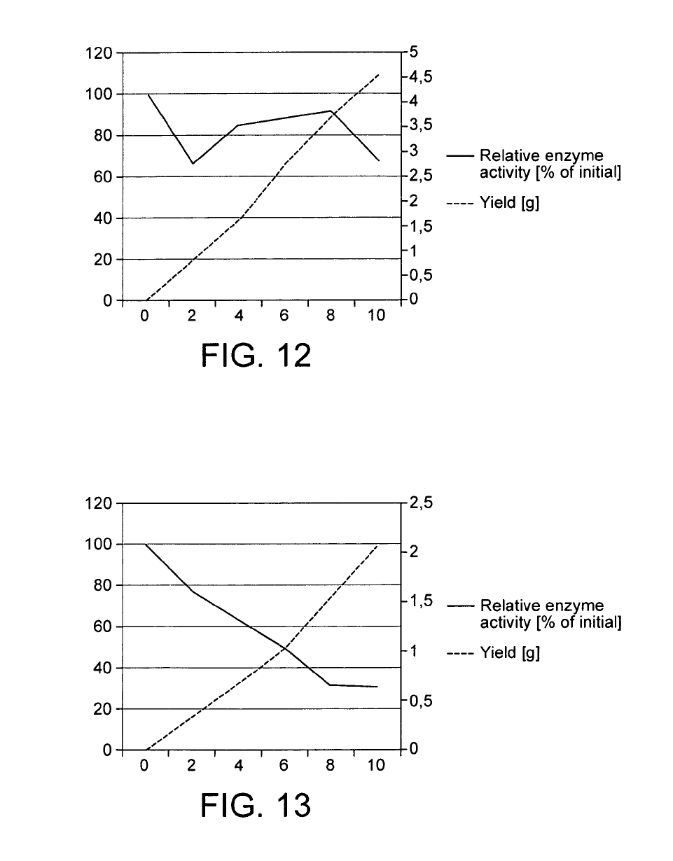

As shown in FIG. 9, HPLC analysis shows that the plant protein hydrolysate does not differ significantly from plant protein hydrolysate of the batch process. The top line of FIG. 9 shows filtered wheat gluten hydrolysis product from factory production sample (0.45 μm filtered). The middle line shows batch produced wheat gluten hydrolysis product which was produced for comparison at bench scale (10 kDa filtered). The bottom line shows a ten hour sample from the membrane reactor experiment using a 10 kDa MWCO membrane.

The invention includes the state of the art science and technology for powder wetting, enzyme kinetic understanding (biotransformation), membrane bioreactor technology (Fractionation and Membrane Technology), sensory analysis, and understanding recent trends in consumer market trends research and product application.

The invention provides energy efficiency and operational simplicity, high transport selectivity, large operational flexibility and environment compatibility. The invention also provides means for enhanced molecular separations and chemical transformations overcoming existing limits of the traditional industrial processes.

The advantages of the invention demonstrate that the enzymes applied are still active at the end of the plant protein hydrolysis. In the batch process the enzymes need to be inactivated at the end of the protein hydrolysis. Since the enzymatic (also called bio-catalytic) function is catalytic it is logical that a more efficient use of enzyme can be obtained by retaining or recovering it during or after plant protein hydrolysis.

The invention enables the fractionation and concentration of plant protein hydrolysates according to size. An advantage of plant protein hydrolysates is the perception by the consumer especially in the case of the vegetarian consumer who would prefer not to consume animal derived protein hydrolysates. Furthermore, the plant protein hydrolysates are manufactured in a natural way.

As used in this specification, the words “comprises”, “comprising”, and similar words, are not to be interpreted in an exclusive or exhaustive sense. In other words, they are intended to mean “including, but not limited to”.

Further, any reference in this specification to prior art documents is not intended to be an admission that they are widely known or form part of the common general knowledge in the field.

### EXAMPLES

The invention is further described with reference to the following examples. It will be appreciated that the invention as claimed is not intended to be limited in any way by these examples.

### Example 1

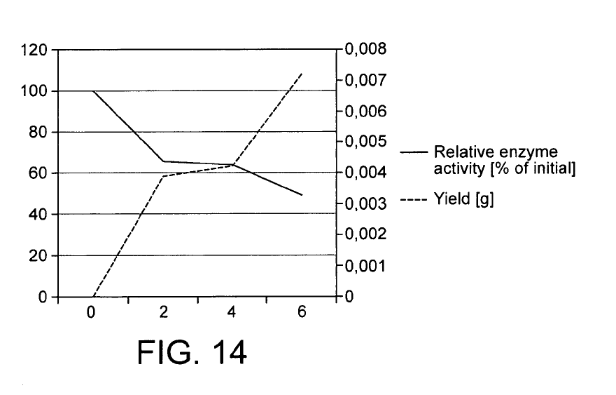

In this example the following process parameters of a double loop enzyme membrane system operated in semi-continuous mode (removal of 600 mL filtrate every 2 hours): Trans membrane pressure 3±0.5 bar, membrane velocity 4-6 m/s, reactor volume 2 L, Flavourzyme™: 1 g/L Wheat gluten 10 g/L, pH 5, T 50° C. The yield and enzyme stability in this experiment are shown in FIG. 10.

### Example 2

in this example the following process parameters of a double loop enzyme membrane system operated in semi-continuous mode (removal of 600 mL filtrate every 2 hours): Trans membrane pressure 3±0.5 bar, membrane velocity 4-6 mils, Reactor volume 2 L, Flavourzyme™: 5 g/L wheat gluten 10 g/L, pH 5, T 40° C. The yield and enzyme stability in this experiment are shown in FIG. 11.

### Example 3

In this example the following process parameters of a double loop enzyme membrane system operated in semi-continuous mode (removal of 600 mL filtrate every 2 hours): Trans membrane pressure 3±0.5 bar, membrane velocity 4-6 m/s, Reactor volume 2 L, Flavourzyme™: 5 g/L, wheat gluten 20 g/L, pH 7, T 50° C. The yield and enzyme stability in this experiment are shown in FIG. 12. The relatively high yield can be explained by the fact that an optimal enzyme substrate ratio was applied in combination with good conditions for enzyme activity and stability.

### Example 4

In this example the following process parameters of a double loop enzyme membrane system operated in semi-continuous mode (removal of 600 mL filtrate every 2 hours): Trans membrane pressure 3±0.5 bar, membrane velocity 4-6 mils, reactor volume 2 L, Flavourzyme™: 5 g/L, wheat gluten 20 g/L, pH 5, T 30° C. The yield and enzyme stability in this experiment are shown in FIG. 13.

### Example 5

In this example the following process parameters of a double loop enzyme membrane system operated in semi-continuous mode (removal of 600 mL filtrate every 2 hours): Trans membrane pressure 3±0.5 bar, membrane velocity 4-6 mils, Reactor volume 2 L, Flavourzyme™: 0.1 g/L, wheat gluten 20 g/L, pH 7, T 30° C. The yield and enzyme stability in this experiment are shown in FIG. 14. Due to the relatively low enzyme activity compared to the relatively high substrate load, this experiment had to be stopped after 6 hours due to membrane blockage by substrate.

It is to be appreciated that although the invention has been described with reference to specific embodiments, variations and modifications may be made without departing from the scope of the invention as defined in the claims. Furthermore, where known equivalents exist to specific features, such equivalents are incorporated as if specifically referred to in this specification.

1. A membrane reactor for the manufacture of plant protein hydrolysates, the membrane reactor comprising:
a substrate vessel adapted to provide a plant protein substrate to an enzyme source;
a continuously stirred reactor comprising the enzyme source; and
an ultrafiltration module comprising a membrane with a molecular cut-off wherein the membrane is adapted to allow passage of the plant protein hydrolysate while retaining the enzyme.

2. The membrane reactor as claimed in claim 1, further comprising:
a first circulation loop enabling a mixture of the plant protein substrate and enzyme source to be transferred from the continuously stirred reactor to the ultrafiltration module and at least some of the mixture to be returned to the continuously stirred reactor; and
a second circulation loop enabling the mixture received from the first circulation loop to be circulated through or over the membrane and at least some of the mixture to be returned to the first circulation loop.

3. The membrane reactor as claimed in claim 2, wherein the first circulation loop operates at or close to atmospheric pressure and the second circulation loop operates at a pressure of 1 to 8 bar.

4. The membrane reactor as claimed in claim 1, comprising a heating device adapted to maintain a temperature of the content of the continuously stirred reactor between 25° C. and 75° C.

5. The membrane reactor as claimed in claim 1, comprising an electro dialysis system.

6. The membrane reactor as claimed in claim 1, comprising a separation device, capable of separating insoluble matter from the plant protein hydrolysate.

7. The membrane reactor as claimed in claim 1, wherein the membrane has a pore size of 1 to 20 nanometres.

8. The membrane reactor as claimed in claim 1, wherein the enzyme is selected from the group consisting of an endopeptidase, an exopeptidase, a glutaminase, or an enzyme derived from Basidiomycetes, and combinations thereof.

9. A method for the manufacture of plant protein hydrolysates for food stuffs comprising using a membrane reactor for the manufacture of plant protein hydrolysates, the membrane reactor comprising a substrate vessel adapted to provide a plant protein substrate to an enzyme source, a continuously stirred reactor comprising the enzyme source, and an ultrafiltration module comprising a membrane with a molecular cut-off wherein the membrane is adapted to allow passage of the plant protein hydrolysate while retaining the enzyme.

10. A method for the manufacture of plant protein hydrolysates for use in food, the method comprising:
providing a suspension of plant protein;
adding to the suspension of plant protein an enzyme to form a mixture such that plant protein hydrolysis occurs;
filtering the resulting mixture through an ultrafiltration module comprising a membrane with a molecular cut-off; and
collecting the filtrate comprising the plant protein hydrolysate for use as a food.

11. The method as claimed in claim 10, wherein the filtering comprises circulating the mixture between a continuously stirred reactor and an ultrafiltration module such that some of the mixture is returned from the ultrafiltration module to the continuously stirred reactor and some of the mixture is circulated through or over the membrane.

12. The method as claimed in claim 10, wherein the suspension of plant protein comprises 0.5 to 50% (w/w) plant protein.

13. The method as claimed in claim 10, wherein the plant protein is derived from wheat, soy, corn, potato, pea or cassava.

14. The method as claimed in claim 10, wherein the enzyme is selected from the group consisting of an endopeptidase, an exopeptidase, a glutaminase, or an enzyme derived from Basidiomycetes, and combinations thereof.

15. The method as claimed in claim 10, wherein prior to filtration, insoluble material is separated from the mixture.

16. A plant protein hydrolysate obtainable by a method for the manufacture of plant protein hydrolysates for use in food, the method comprising:
providing a suspension of plant protein;
adding to the suspension of plant protein an enzyme to form a mixture such that plant protein hydrolysis occurs;
filtering the resulting mixture through an ultrafiltration module comprising a membrane with a molecular cut-off; and
collecting the filtrate comprising the plant protein hydrolysate for use as a food, wherein the plant protein hydrolysate is a taste enhancing compound for use in foodstuffs.
